(19) United States
(12) Patent Application Publication
(10) Pub. No.: US 2024/0182947 A1
(43) Pub. Date: Jun. 6, 2024

(54) METHODS OF PREPARING AFLATOXIN CONTAMINATED NUTS, AND USES AND PRODUCTS THEREOF

(71) Applicant: OLAM AMERICAS, LLC, Fresno, CA (US)

(72) Inventors: Jacob Sebastian, Oak Park, IL (US); John Martin, Oak Park, IL (US)

(21) Appl. No.: 18/526,884

(22) Filed: Dec. 1, 2023

Related U.S. Application Data
(60) Provisional application No. 63/429,739, filed on Dec. 2, 2022.

(51) Int. Cl. C12Q 1/24; A23B 9/30; A23L 25/00; C12N 1/14

(52) U.S. Cl. CPC C12Q 1/24; A23B 9/30; A23L 25/20; A23L 25/40; C12N 1/14

(57) ABSTRACT

The disclosure provides methods of preparing aflatoxin-contaminated nuts.

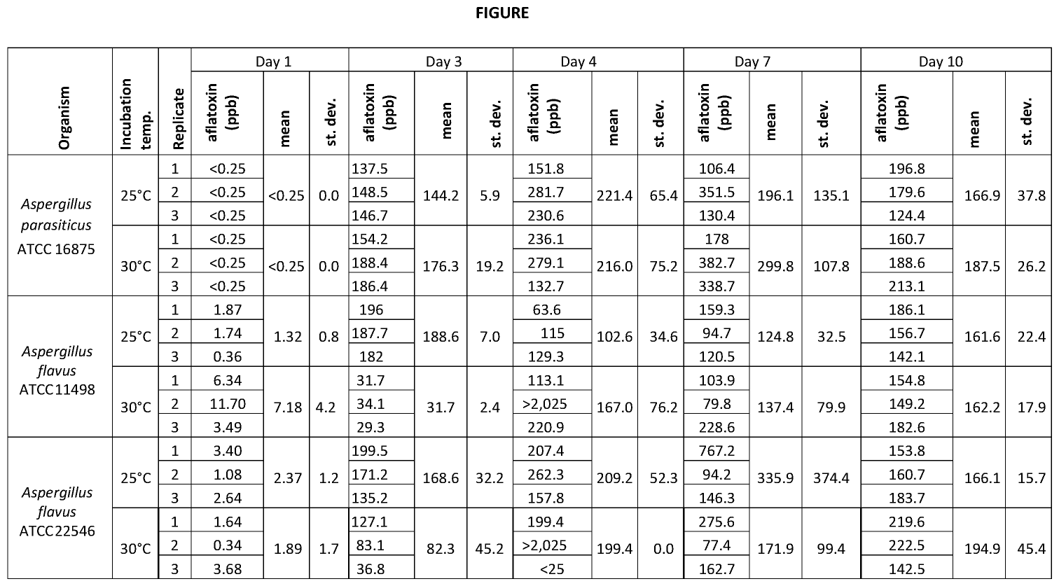

### CROSS-REFERENCE TO RELATED APPLICATIONS

The present application claims the priority benefit of U.S. Provisional Application No. 63/429,739, filed on Dec. 2, 2022, which application is hereby incorporated herein by reference in its entirety.

### FIELD

This disclosure relates to methods of preparing aflatoxin- and/or Aspergillus-contaminated foodstuff, such as nuts.

### TECHNICAL FIELD

Aflatoxins are a group of structurally related mycotoxins. Aflatoxins are produced by specific Aspergillus species and can be toxic, mutagenic, and/or carcinogenic. Aspergillus flavus and Aspergillus parasiticus are the main toxigenic species. A. flavus is not host-specific and infects a variety of food crops, while A. parasiticus is more host specific, and in particular, may contaminate peanuts. See e.g., Jallow et al, 2021, Compr Rev Food Sci Food Saf. 20: 2332-2381).

Aflatoxin contamination and fungal invasion are found in agricultural products and foods, such as nuts, cocoa, and spices, and consequently pose a large problem for consumer safety. Supply chain detection practices can mitigate consumer exposure but are accompanied by substantial economic losses since contaminated food products are destroyed. Methods of decontamination are disclosed in the prior art. However, it can be difficult to compare and evaluate results of different decontamination methods, when the starting material has a wide and variable range of contamination.

The present disclosure is directed to methods of preparing aflatoxin- and/or Aspergillus-contaminated nuts.

### SUMMARY

The method provides a method of preparing nuts having a specifiable aflatoxin contamination level. The method enables a reliable source of aflatoxin-contaminated nuts, and permits in situ production. The contaminated nuts prepared by the method can be used, for instance, to test, develop, and/or optimize methods of decontamination in a more controlled manner, compared to naturally-contaminated nuts.

### BRIEF DESCRIPTION OF THE DRAWINGS

The accompanying drawing is incorporated in and constitute a part of this specification and illustrate various methods and compositions disclosed herein.

The FIGURE depicts data related to three different Aspergillus strains tested for growth and aflatoxin production on clean hazel nuts using two sets of incubation conditions and six timepoints, as described in Example 1.

### DETAILED DESCRIPTION

### Definitions & Abbreviations

Unless defined otherwise, all technical and scientific terms used herein generally have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +/−10%, from the specified value, as such variations are appropriate to perform the disclosed methods.

As used herein, the term “aflatoxin” refers to a chemical substance that is part of a group of structurally related mycotoxins that are produced by certain species of fungi, such as Aspergillus flavus and Aspergillus parasiticus. Aflatoxins can be toxic, mutagenic and/or carcinogenic. Aflatoxins from the B-series (aflatoxins B1 and B2), the G-series (aflatoxins G1 and G2), and M-series are some of the main types of interest and are regulated for safety of food and agricultural products. Aflatoxin B1 (AFB1) is the most potent carcinogen of the aflatoxins. Aspergillus flavus and Aspergillus parasiticus are closely related. Aspergillus flavus typically produces B1 and B2 and A. parasiticus typically produces G1, G2, and M1, as well as B1 and B2. (Diener et al., 1987, “Epidemiology of aflatoxin formation by Aspergillus flavus.” Annual review of phytopathology 25(1): 249-270).

As used herein, “nut” refers to an edible nut. Exemplary edible nuts include, but are not limited to, almonds, cashews, hazelnuts, macadamias, peanuts, pecans, pistachios, and walnuts. In the methods of the disclosure, nuts can include their shells (shelled nuts) or can have their shells removed (unshelled nuts). Crushed unshelled nuts, including but not limited to, cracked nuts, sliced nuts, nut butters, and nut pastes, can also be artificially contaminated with aflatoxin of at least about 100 parts-per-billion (ppb) and/or presence of Aspergillus flavus and/or Aspergillus parasiticus in an amount resulting in at least about 100 ppb aflatoxin in the methods of the disclosure.

As used herein, “aflatoxin contamination” refers to the presence of one or more aflatoxins present on at least the surface of a nut. As used herein, the surface refers to the shell of a nut (a shelled nut) and to the surface of a nut without a shell (an unshelled nut). As used herein, surface also encompasses surface exposed in abraded nuts, crack nuts, sliced nuts, and the like.

As used herein, the terms “reduction of aflatoxin contamination”, “reducing aflatoxin contamination”, and “aflatoxin reduction” refers to reducing the amount of aflatoxin, such as AFB1, detectable in a foodstuff, such as a nut, or a plurality of nuts. The terms encompass decontamination, detoxification, and both decontamination and detoxification. Decontamination refers to the physical removal of the aflatoxin and/or removal of toxigenic Aspergillus strains, such as Aspergillus flavus and Aspergillus parasiticus. Detoxification refers to the degradation of the aflatoxin, such as AFB1, and/or Aspergillus flavus and Aspergillus parasiticus.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It is understood that any and all whole or partial integers between any ranges set forth herein are included herein.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

Finally, the steps of all methods described herein are performable in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. Numerous modifications and adaptations will be readily apparent to those skilled in this art without departing from the spirit and scope of the invention.

### DESCRIPTION

Embodiments of the present invention are described below. It is, however, expressly noted that the present invention is not limited to these embodiments, but rather the intention is that modifications that are apparent to the person skilled in the art and equivalents thereof are also included.

This disclosure is generally related to a method of preparing aflatoxin-contaminated nuts. In an embodiment, the method is directed to preparing Aspergillus-contaminated nuts and incubating the nuts under conditions to permit production of aflatoxin. The method can yield aflatoxin-contaminated nuts at a specifiable extent of contamination in a reasonably short amount of time.

This method comprises: (1) preparing a conidial suspension from a toxigenic Aspergillus strain, (2) inoculating nuts with the conidial suspension, and (3) incubating the inoculated nuts, wherein said Aspergillus strain grows and produces at least one aflatoxin.

Any toxigenic Aspergillus mold can be used in the method. In an embodiment, the toxigenic Aspergillus is Aspergillus parasiticus. In an embodiment the Aspergillus parasiticus is Aspergillus parasiticus ATCC 16875. In an embodiment, the toxigenic Aspergillus is Aspergillus flavus. In an embodiment the Aspergillus flavus is Aspergillus flavus ATCC 11498 or Aspergillus flavus ATCC 22546.

Preparing a fungal conidial suspension is known to the skilled artisan. In general, a fungal conidial suspension is prepared by harvesting mycelia and conidia from plates of actively growing Aspergillus cultures into sterile water (such as autoclaved water). Harvesting can be done by scraping and/or swabbing the actively growing cultures. Conidia can be pelleted by centrifuge and resuspended in an appropriate aqueous solution, such as sterile deionized water containing 0.1% Tween. Conidial counts are assessed by methods known in the art, and preferably are assessed in triplicate for a conidial suspension to be used in the method of the disclosure. A conidial suspension having about 10⁴ to 10⁷ colony-forming units per milliliter (CFU/ml) is useful in the practice of the method. In an embodiment, a conidial suspension having about 10⁵ colony-forming units per milliliter (CFU/ml) is used to prepare contaminated nuts.

The culture conditions for growing Aspergillus should be selected for sufficient sporulation and growth, in order to prepare a suitably concentrated conidial suspension. Aspergillus strains can be cultured on different culture media to identify a suitable media. Examples of culture media for Aspergillus are malt-extract agar (MEA); Potato Dextrose Agar (PDA); and Aspergillus flavus/parasiticus agar (AFPA). Other culture media can be used.

Any edible nut can be used in the method. The nuts can be shelled or unshelled. Exemplary edible nuts include, but are not limited to, almonds, cashews, hazelnuts, macadamias, peanuts, pecans, pistachios, and walnuts. In an embodiment, the nuts are unshelled hazelnuts.

To inoculate nuts with a conidial suspension of an Aspergillus strain, the nuts are treated reduce levels of natural microflora/mycoflora by known methods. For instance, the nuts can be immersed the nuts in 1% sodium hypochlorite for an amount of time to reduce natural contamination. In an embodiment, the nuts are immersed in 1% sodium hypochlorite for about 10 minutes. The nuts are then rinsed with sterile water (such as sterile distilled water) and the residual water is removed, for instance by contact with absorbent paper. Optionally, prior to treatment to reduce levels natural microflora/mycoflora, the initial water activity and moisture of the nuts can be assessed. The water activity and moisture can be monitored during the process of preparing contaminated nuts, and steps optionally can be taken to restore nuts to the original levels, for instance, by maintaining nuts in a humidified environment.

The nuts are inoculated by contacting nuts with a conidial suspension prepared from a toxigenic Aspergillus strain. The nuts can be in a sterile sealable container and an appropriate amount of a conidial suspension is added to the container. The nuts and conidial suspension are manipulated in order to distribute the inoculum substantially equally over the entire quantity of nuts. For instance, the washed nuts can be placed into a sterile bag. For monitoring purposes, inoculated nuts can be prepared in triplicate. For instance, three 1100 grams of nuts are placed into separate sterile bags. A specific quantity of conidial suspension is added to each sterile bag of nuts and mixed thoroughly to distribute the inoculum over the nut sample.

After dispersing the inoculum over the nuts, the inoculum-treated nuts may be dried. For instance, the nuts may be transferred to trays lined with filter paper and dried at ambient temperature under laminar flow of filtered air. However, drying should be controlled to minimize possible adverse effect on the spores, such as desiccation that may be lethal to the spores. After inoculation, the water activity of the nuts can be checked periodically until it has returned to pre-inoculation levels. The inoculated nuts can be analyzed to determine initial inoculum levels.

After inoculation of the nuts, the nuts are maintained in an appropriate environment to permit the growth of Aspergillus and production of aflatoxin. An appropriate environment can be a sealed humidified container, having a relative humidity of >90%, and maintained at 25° C.±2° C. to 30° C.±2° C. For instance, inoculated nuts can be distributed in a single layer on a mesh tray above water in a temperature controllable, sealable container maintained at relative humidity of >97%. To ensure consistent conditions, the humidified container can be preconditioned to the temperature and relative humidity level intended for the incubation of the inoculated nuts. The inoculated nuts are maintained in the appropriate environment for a sufficient amount of time for aflatoxin development. The incubation duration can vary based on the Aspergillus strain used for inoculation. The time can be from 2 days to 10 days, 3 days to 10 days, or 4 days to 7 days. The time can be about 4 days. In an embodiment, inoculated nuts are maintained at 30° C.±2° C. for about 4 days at a relative humidity of >97%.

After incubation, aflatoxin level is measured. Aflatoxin can be measured on a washed sample of nuts to determine resilience of contamination. Different wash conditions can be used. For instance, a first wash condition can be washing the nuts with water for 10 minutes. Another wash condition can be washing the nuts is 1% sodium hypochlorite (NaOCl) for 10 minutes, followed by water rinse. The washed nuts are dried until their water activity/moisture has returned to pre-inoculation levels.

Aflatoxin level is measured on a sample of the inoculated nuts after incubation. Methods of measuring aflatoxin are known to the skilled artisan. See, e.g., Miklós et al., 2020, Detection of Aflatoxins in Different Matrices and Food-Chain Positions. Front Microbiol. 11:1916. Erratum in: Front Microbiol. 2021 May 13; 12:669714. Methods generally comprise extracting aflatoxin from a nut sample and the quantifying the aflatoxin. An exemplary method comprises grinding a nut sample using a grinder, adding methanol and stirring to for a smooth slurry. The slurry is filtered, e.g., through Whatman filter paper, and diluted with distilled water. The diluted slurry is passed over an aflatoxin-immunoaffinity column, such as R-Biopharm, R5001, and then washed. The bound aflatoxin is eluted and the quantity of aflatoxin is assessed by, for instance, an ELISA test (e.g., commercially available products) or using high performance liquid chromatography (HPLC) method and fluorescence detector.

Aflatoxin level can be measured periodically during incubation. For instance, samples of incubated nuts can be tested at days 2, 3, and 4 of incubation to monitor development of aflatoxin.

An exemplary non-limiting protocol for preparing Aspergillus-contaminated nuts is as follows.

### Preparation of Aspergillus

1. Prepare 10×90 mm Malt Extract Agar (MEA) plate cultures of Aspergillus flavus (ATCC #11498; CABI #52140), Aspergillus parasiticus (ATCC #16875; CABI #124931), and Aspergillus flavus (ATCC #22546; CABI #370082). Although CABI #124931 is designated as Aspergillus parasiticus in their catalog, ATCC #16875 is designated Aspergillus flavus. Catalog numbers for Aspergillus flavus strain ATCC 16875 in different collections include: CBS 573.65; IFO 7540; QM 6738; WB 500; and NRRL 500.
2. Incubate plates at 25° C. for 10 days.
3. Examine microscopically to confirm formation of conidia.
4. In a biological safety cabinet, harvest conidia by swabbing and resuspend in 20 ml sterile deionized water with 0.1% Tween (SDWT).
5. Transfer conidial suspensions into centrifuge tubes and pellet conidia by centrifugation (4,000 rpm, 10 minutes).
6. Discard supernatant and resuspend conidial pellets in 20 mL SDWT.
7. Estimate conidial counts using a Neubauer counting chamber (WI-MB-11-025) and confirm by enumerating conidial suspension in triplicate on Aspergillus flavus and parasiticus agar (AFPA).
8. Adjust levels to 10⁵ CFU/mL.
9. Store suspension at 2-8° C. until required (up to 1 week).

### Preparation of Nuts

1. Determine initial water activity and moisture levels of the hazelnuts.
2. Immerse samples in 1% sodium hypochlorite solution for 10 minutes to reduce levels of natural microflora/mycoflora.
3. Rinse with sterile distilled water to remove residual hypochlorite.
4. Remove most of residual water with absorbent paper.
5. If including damaged hazelnuts in process, first place 1100 g of hazelnuts into a cement mixer and operate for 3 hours.

### Inoculation of Nuts

1. Place 3×1,100 g hazelnut samples into large sterile stomacher bags.
2. Add ˜15 ml of A. parasiticus or A. flavus conidial suspension to each bag and mix thoroughly by hand to evenly distribute the inoculum over the sample.
3. Transfer into trays lined with filter paper and dry at ambient temperature under laminar flow of filtered air.
4. Check water activity periodically until it has returned to pre-inoculation levels.
5. Analyze triplicate samples to determine initial inoculum levels achieved after drying.

### Development of Aflatoxin

1. Transfer samples into sealed 50 L boxes containing 15 L sterile deionized water. Place nut samples onto a mesh tray in a single layer and suspend above the water at relative humidity >97%. Confirm humidity using a calibrated humidity meter.
2. Incubate samples at 30+2° C. for 4 days.
3. After incubation, take a small sample from each batch and image under UV light. Use uninoculated hazelnuts as a control image.
4. Take a sample of nuts immediately after incubation, wash (two conditions) and measure aflatoxin level to determine resilience of contamination.
   Wash condition 1: Water, 10 minutes
   Wash condition 2: 1% hypochlorite, 10 minutes, followed by water rinse
5. Spread out nuts in trays and dry them until their water activity/moisture has returned to pre-inoculation levels.
6. Take 5×50 g samples from each batch and analyze to determine aflatoxin levels using r-Biopharm® total aflatoxin ELISA kit (R-Biopharm AG, Darmstadt, Germany) with aflatoxin immunoaffinity columns.

Another method of obtaining aflatoxin-contaminated-nuts is to contact nuts with an aflatoxin solution comprising at least one purified aflatoxin at a known concentration in a suitable solvent, such as methanol. To prevent degradation, the aflatoxin solution can be stored in amber glass containers in a refrigerator. Nuts can be cleaned to reduce the present of naturally occurring flora, as discussed above. The clean nuts are then contacted with the aflatoxin solution and manipulated to obtain a substantially homogenous distribution of the solution over the nut surfaces. The coated nuts are then dried, for instance, for 24 hours in the dark, to allow the methanol to evaporate. The amount of aflatoxin on the contaminated nuts can be assessed by methods known in the art, as discussed above. Contaminated nuts prepared according to this methods and stored in ambient storage in dark conditions maintain the aflatoxin contamination level for at least 21 days. These contaminated nuts lack the present of mold structure and naturally-produced mixtures of aflatoxins that are produced by the Aspergillus-contaminated nuts method.

### EXAMPLES

The inventors sought to generate generating aflatoxin-contaminated nuts for use in validation studies of aflatoxin-mitigation techniques. The inventors undertook an exploration of the materials and conditions with the goal of reliably generating contaminated nuts having, for instance, approximately 200 ppm total aflatoxin. Two overall approaches were tried. One approach was to inoculate nuts with a toxigenic Aspergillus strain under controlled conditions to allow Aspergillus growth and toxin formation. This approach is desirable because the present of mold structures is expected to better mimic real-world contamination. Another approach was directly spiking nuts with a standard aflatoxin B1 solution, resulting in nuts with a surface coating of aflatoxin without mold structures.

### Example I: Aspergillus-Inoculated Nuts

This approach entailed seeding nuts with Aspergillus mold spores and allowing the spores to grow and produce aflatoxins naturally.

### Identification of Aspergillus Strains and Conidial Suspension Preparation

Three toxigenic Aspergillus strains were selected based on their ability to produce aflatoxins. See Table 1.

TABLE 1

| | Strain Reference | ATCC | |
|---|---|---|---|
| | Number (CABI; | reference | Aflatoxins |
| Species name | formerly IMI) | number | produced |
| Aspergillus flavus | IMI 370082 | ATCC 22546 | B1 an B2 |
| Aspergillus | IMI 124931* | ATCC 16875 | B1 and B2 |
| parasiticus | | | |
| Aspergillus flavus | IMI 52140 | ATCC 11498 | B1, B2, |
| | | | G1, and G2 |

*CABI lists this strain as Aspergillus parasiticus. However, ATCC and other collections designate it as Aspergillus flavus (CBS 573.65; IFO 7540; QM 6738; WB 500; BRRL 500.) It may be that the strain has been miscategorized by CABI.

Aflatoxigenic Aspergillus strains were screened for aflatoxin production, based on the method of Davis et al. (1966, Production of aflatoxins B1 and G1 by Aspergillus flavus in a semisynthetic medium. Appl Microbiol. 1966 May; 14(3):378-80.) Strains were cultured on malt-extract agar (MEA) at 25° C. for 10 days until mature conidia formed. Conidia were harvested by swabbing the plates. The conidia were resuspended in sterile deionized water containing 0.1% Tween (SDWT). For each strain, 1 milliliter (ml) of spore suspension was added to Yeast Extract Sucrose (YES) medium (20% sucrose, 2% yeast extract). Cultures were incubated for 7 day at 25° C. as stationary cultures. After incubation, the cultures were filtered through sterile glass wool to remove fungal mycelial fragments. Aflatoxin level in the filtrates was assessed using the R-Biopharm Total Aflatoxin kit R4701 (RIDASCREEN® Aflatoxin Total R4701; R-Biopharm, Darmstadt, Germany). The data are shown in Table 2. The level of aflatoxin for A. flavus strain 11498 was below the limit of quantification for the assay.

TABLE 2

| | ATCC reference | Aflatoxin |
|---|---|---|
| Species name | number | concentration (ppb)d |
| Aspergillus flavus | ATCC 22546 | 124.8 |
| Aspergillus parasiticus | ATCC 16875 | 131.1 |
| Aspergillus flavus | ATCC 11498 | <1.75 |

The initial experiments to prepare Aspergillus-contaminated nuts were then performed utilizing the three Aspergillus strains. The nuts were hazelnuts. Each strain was tested for growth and aflatoxin production on clean hazelnuts using two sets of incubation conditions and six timepoints. Temperatures tested were 25° C. and 30° C. and timepoints over 0-10 days were tested. Humidity was fixed at >97% relative humidity (RH).

### Preparation of Nuts

Initial water activity and moisture levels of the hazelnuts were determined. Nut samples (400 grams each) were immersed in 1% sodium hypochlorite (NaOCl) for 10 minutes to decontaminate the nuts by reducing the levels of naturally-occurring flora. The decontaminated nuts were rinsed with sterile distilled water (SDW) to remove residual sodium hypochlorite. The rinsed nuts were dried on paper towels. The nuts were then transferred into trays lines with filter paper. The trays were placed in racks and the nuts were dried at 20° C. in 30 m³ chamber containing dehumidifiers with humidistats set to 30% relative humidity (RH).

### Inoculation of Nuts

For each of the three test strains, 2×400 gram nut samples were placed into large sterile bags and surface-inoculated by adding about 15 ml of Aspergillus conidial suspension described above to each bag, and mixing thoroughly by hand to evenly distribute the inoculum over the sample. The samples were then transferring into trays lined with filter paper and dried as described above. Water activity was checked periodically until it returned to pre-inoculation levels. Samples were analyzed to determine initial inoculum levels present after drying.

### Development of Aflatoxin

For each of the three test strains, 2×400 gram inoculated nut samples were transferred into sealed boxes (ThermoScientific™ AnaeroPack™ 7.0 L Rectangular Jar, Thermo Fisher Scientific, Waltham, MA) containing 2 liters of sterile distilled water (SDW) to maintain relative humidity level of >97%. Samples were suspended above the water in the humid air. The samples were incubated at 25° C. and 30° C. These conditions were chosen as suitable conditions for aflatoxin production on nuts (Arrus et al, 2005, Aflatoxin production by Aspergillus flavus in Brazil nuts, Journal of Stored Products Research, 41(5): 513-527; Diener et al., 1967, Limiting temperature and relative humidity for growth and production of aflatoxin and free fatty acids by Aspergillus flavus in sterile peanuts. J Am Oil Chem Soc. 44(4):259-263).

At days 1, 3, 4, 7, and 10 of incubation, triplicate samples were taken to measure the levels of aflatoxin (ng/g=ppb) and log colony forming units per gram (log CFU/g) of aflatoxigenic Aspergillus species on the hazelnut samples. Aflatoxins were extracted by homogenizing 18 g samples using a Waring grinder. Five gram (5 g) samples of homogenate were transferred to sterile containers and 25 ml of 70% methanol was added to each sample. The samples were homogenized using an overhead homogenizer at full speed for 2 minutes to form a smooth slurry. The slurries were filtered through Whatman #1 filter paper. Then, 5 ml of the filtrate was diluted in 15 ml of glass-distilled water (GDW), and the resulting 20 ml sample was passed over a RIDA® Aflatoxin column R5001 (R-Biopharm, Darmstadt, Germany), washed, and the aflatoxins eluted and diluted 1 in 10 in GDW in according with the manufacturer's instructions. Aflatoxin quantity of the diluted purified extracts was assessed using RIDASCREEN® Aflatoxin Total R4701 (R-Biopharm, Darmstadt, Germany), using duplicate ELISA wells; ELISA tests were conducted in according with the manufacturer's instructions.

To assess log CFU/g counts of aflatoxigenic Aspergillus species, at each sampling point (i.e., day 1, day 3 etc.), a further 10 g sample of nuts was taken and placed into a sterile stomacher bag and 90 ml of Maximum Recovery Diluent (MRD) was added. Samples were shaken in a pulsifier for 2 minutes at full power to obtain a suspension of fungi. The fungi suspensions were serially diluted in further 9 ml aliquots of MRD, and 0.5 ml aliquots of each dilution were transferred onto duplicate pre-poured Aspergillus flavus/parasiticus agar (AFPA) plates and spread using a sterile spreader. Plates were inverted and incubated at 30° C. for 3-5 days, and typical colonies (colonies with orange reverse sides) were counted and used to calculate log CFU/g counts in each sample. The log CFU/g count data are shown in Table 3.

TABLE 3

| | | Log CFU/g count | | | | | |
|---|---|---|---|---|---|---|---|
| Organism | Temperature | Day 0 | Day 1 | Day 3 | Day 4 | Day 7 | Day 10 |
| A. flavus | 25° C. | 3.69 | 2.78 | 6.43 | 5.82 | 5.79 | 7.07 |
| ATCC 22546 | 30° C. | 3.69 | 3.81 | 4.60 | 5.83 | 5.90 | 6.81 |
| A. parasiticus | 25° C. | 1.00 | 1.81 | 5.31 | 4.88 | 6.04 | 6.27 |
| ATCC 16875 | 30° C. | 1.00 | 1.73 | 5.34 | 6.35 | 6.69 | 6.93 |
| A. flavus | 25° C. | 3.73 | 2.91 | 3.85 | 4.08 | 5.64 | 5.69 |
| ATCC 11498 | 30° C. | 3.73 | 2.51 | 4.56 | 4.05 | 5.52 | 5.64 |

The aflatoxin concentration data are shown in the FIGURE. Aspergillus flavus 11498 did not reach 200 ppb aflatoxin production under any conditions. All three strains appeared to converge on 150 to 200 ppb aflatoxin after 10+ days, at both temperatures. Aspergillus parasiticus 16875 and Aspergillus flavus 22546 both reached 200 ppb after 4 days at both temperatures, indicating that these strains have reasonable temperature range resilience. The amount of aflatoxin for these two strains was fairly stable from day 4 to day 10.

Aspergillus parasiticus was used in scaled-up amounts intended to further explore certain aspects of the inoculation method steps, including physically damaging nuts prior to inoculation and the impact of the decontamination step. However, the aflatoxin levels and the log CFU/g counts after incubation at >97% RH, at 30° C. for 4 days in the scaled-up method were unexpectedly very low, compared to what was predicted based on the initial experiments. Possible reasons for the low levels include: (1) weak growth/sporulation of A. parasiticus in the initial culture to produce inoculum; (2) lethal desiccation of spores during the post-inoculation drying step; and (3) the chamber used for incubation did not reach >97% RH until 19 hours into the incubation period.

A revised protocol was designed and tested again using Aspergillus parasiticus 16875. The amended method was also practiced using Aspergillus flavus 22568 which shows stronger sporulation and growth. To examine the growth and sporulation in the initial culture to produce inoculum, cultures of Aspergillus parasiticus 16875 and Aspergillus flavus 22568 were prepared on three different culture media: malt-extract agar (MEA); Potato Dextrose Agar (PDA); and AFPA, for 10 days at 25° C. Cultures on MEA showed the highest degree of sporulation and were therefore harvested, filtered, and centrifuged to concentrate conidia as described above. Two 1.1 kilogram (kg) batches of hazelnuts were treated with 1% sodium hypochlorite for 10 minutes and rinsed with sterile distilled water (SDW) to remove residual sodium hypochlorite. The rinsed nuts were dried on paper towels, and then transferred into trays lines with filter paper. The trays were placed in racks and the nuts were dried at 20° ° C. in 30 m³ chamber containing dehumidifiers with humidistats set to 30% relative humidity (RH).

One 1.1 kg batch of hazelnuts was inoculated with Aspergillus parasiticus 16875 and the second 1.1 kg batch of hazelnuts was inoculated with Aspergillus flavus 22568, as described above, except that no drying step was applied after inoculation of the nuts.

In addition, the humidified growth chambers were pre-conditioned by incubation at 30° C. for 24 hours prior to addition of the inoculated nuts.

After 4 days incubation at 30° C. and >97% R, the aflatoxin levels and the log CFU/g levels were assessed, as described above. Aflatoxin levels achieved in the hazelnuts after 4 days incubation at 30° C. and >97% RH were substantially higher following the revised protocol.

TABLE 4

| | Mean aflatoxin level (ppb) | Standard |
|---|---|---|
| Organism | (5 replicates) | deviation |
| A. flavus 22568 | 143.9 | 83.4 |
| A. parasiticus 16875 | 140.0 | 46.7 |

Log CFU/g counts of aflatoxigenic molds of 6.69+0.53 CFU/g and 6.01+0.09 CFU/g were determined for A. flavus 22568 and A. parasiticus 16875, respectively.

These data are more consistent to what was predicted based on the initial studies. The data support the importance of obtaining a high degree of sporulation for use in the conidial suspension used to inoculate the nuts. In addition, these data support the importance of minimizing steps that may cause lethality of the Aspergillus spores, as well as incubating the inoculated nuts in stable conditions of temperature and humidity to obtain reliable, consistent aflatoxin-contaminated nuts in practicing the method.

### Example 2: Direct Contamination of Nuts with Aflatoxin B1

This approach entailed contacting nuts with a solution of aflatoxin at a known concentration.

Aflatoxin B1 was obtained from Sigma Aldrich (A6636, 98% purity, 5 mg). Aflatoxin was dissolved in 20 ml methanol to produce a 250 microgram per milliliter (μg/ml) stock solution. The solution was stored under refrigerated conditions in an amber glass container.

A 10 μg/ml spiking solution was prepared by adding 0.8 ml of the 250 μg/ml stock solution to 20 ml methanol in an amber volumetric flask. The 10 μg/ml solution was used to spike hazelnuts to 200 μg/kg (ppb) as follows. One hundred grams of clean hazelnuts were placed into a sterile 4 L bag and 2 ml of the 10 μg/ml spiking solution was added. The bag was sealed then shaken and manually manipulated to ensure homogenous distribution of the solution over the hazelnut surfaces. Samples were transferred to a 140 mm Petri dish lid lined with filter paper and placed uncovered in a class 1 biosafety cabinet. The uncovered nuts left in the cabinet in the dark for 24 hours to allow complete evaporation of the methanol.

The above process was repeated, with appropriate dilution of the 10 μg/ml spiking solution in methanol to obtain nuts samples with spiking levels of 100 μg/kg (ppb) and 50 μg/kg (ppb) upon addition of 2 ml of the diluted spiking solution.

The spiked hazelnuts were analyzed for aflatoxin levels using HPLC. For each sample, 50 g of spiked nuts were ground into a fine past and blended with 250 ml of 60% methanol/water using an Ultraturrax blender as high speed for 5 minutes. The blended samples were filtered using Whatman filter paper to remove nut residues.

Ten milliliter of filtrate was diluted 1:1 in distilled water and purified using an EASI-EXTRACT® aflatoxin immunoaffinity column (R-Biopharm, Darmstadt, Germany). The column was washed, and the bound aflatoxin was eluted and analyzed by HPLC.

Results of the HPLC analysis data for the hazelnuts spiked to 200 ppb are shown in Table 5.

TABLE 5

| | |
|---|---|
| Day (ambient, | Total aflatoxin level (ppb) |

| dark storage) | Replicate | Batch A | Batch B | Batch C |
|---|---|---|---|---|
| 0 | 1 | 202 | 211 | 273 |
| | 2 | 259 | 199 | 222 |
| | 3 | 243 | 205 | 204 |
| | 4 | 262 | 205 | 187 |
| | 5 | 219 | 224 | 197 |
| | 6 | 252 | 224 | 184 |
| | 7 | 261 | 231 | 211 |
| | 8 | 251 | 230 | 200 |
| | 9 | 217 | 186 | 197 |
| | 10 | 181 | 209 | 192 |
| | mean | 235 | 216 | 195 |
| | Standard | 30 | 15 | 26 |
| | deviation | | | |
| 7 | | 228 | 268 | 230 |
| 14 | | 207 | 203 | 192 |
| 21 | | 202 | 259 | 243 |

These data show that the mean achieved aflatoxin levels showed good uniformity and consistent with the target level. No substantial decrease in aflatoxin level was observed after storage for 7 days, 14 days, and 3 weeks at ambient temperature in dark storage.

The invention is further described in detail by reference to the above experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The disclosures of each and every patent, patent application, and publication cited herein are hereby incorporated herein by reference in their entirety.

Although the present embodiments have been described in detail with reference to examples above, it is understood that various modifications can be made without departing from the spirit of these embodiments, and would readily be known to the skilled artisan. The appended claims are intended to be construed to include all such embodiments and equivalent variations.

What is claimed is:

1. A method to produce aflatoxin-contaminated nuts in vitro, comprising
1) preparing a conidial suspension from a toxigenic Aspergillus strain;
2) inoculating nuts with the conidial suspension; and
3) incubating the inoculated nuts, wherein said Aspergillus mold grows and produces at least one aflatoxin.

2. The method according to claim 1, wherein the toxigenic Aspergillus strain is Aspergillus flavus or Aspergillus parasiticus.

3. The method according to claim 1, further comprising decontaminating the nuts prior to inoculating them.

4. The method according to claim 3, where decontaminating the nuts comprises contacting with sodium hypochlorite, them rinsing the nuts with sterile water.

5. The method according to claim 1, wherein the conidial suspension has 10⁴ to 10⁷ colony-forming units per milliliter (CFU/ml).

6. The method according to claim 1, wherein the conidial suspension has 10⁵ CFU/ml.

7. The method according to claim 1, wherein the step of incubating the inoculated nuts comprises incubating the inoculated nuts at 25° C. to 30° C. in a >97% relative humidity (RH) environment for 2 to 10 days.

8. The method according to claim 1, wherein the step of incubating the inoculated nuts comprises incubating the inoculated nuts at 25° C. to 30° C. in a >97% relative humidity (RH) environment for 4 to 7 days.

9. The method according to claim 1, wherein the nuts are selected from almonds, cashews, hazelnuts, macadamias, peanuts, pecans, pistachios, or walnuts.

10. The method according to claim 1, wherein the incubated inoculated nuts comprise 150 to 300 ppm aflatoxins.

11. The method according to claim 1, wherein the incubated inoculated nuts comprise aflatoxin B1 (AFB1).
